(19) United States
(12) Patent Application Publication
(10) Pub. No.: US 2007/0292468 A1
(43) Pub. Date: Dec. 20, 2007

(54) Coated jewelry articles to reduce skin irritation

(76) Inventors: Joel Studin, Great Neck, NY (US); Robert Giuliano, Eastchester, NY (US)

Correspondence Address: FRENKEL & ASSOCIATES, 3975 UNIVERSITY DR., STE. 330, FAIRFAX, VA, 22030, US

(21) Appl. No.: 11/452,876

(22) Filed: Jun. 14, 2006

(51) Int. Cl. A44C 7/00; A61K 31/573

(52) U.S. Cl. 424/422; 514/171; 63/12

(57) ABSTRACT

Skin irritation is a common problem to those who wear potentially skin irritating jewelry such as pierced earrings. The present invention provides a coated-medicated jewelry article that thwarts skin irritation and may provide relief for irritated skin. The coating contains a film-former and optionally a medicament.

### FIELD OF THE INVENTION

This invention relates to coated jewelry to prevent and/or alleviate skin irritations.

### BACKGROUND OF THE INVENTION

Skin sensitivity can be heightened by constant contact with potentially skin-irritating objects, i.e. watches, rings, bracelets, piercings such as earrings. Such sensitivity is prevalent with pierced earrings worn through pierced ear openings. The discussion of sensitivity to the above-listed skin irritating objects will be exemplified below using the following discussion on pierced earrings.

Numerous people have experienced difficulty and discomfort in wearing earrings suspended from pierced ear openings through the earlobes. One source of problems with pierced earrings has resulted from the sensitivity some people have toward materials that come in contact with the exterior skin surface and/or the tissue within the pierced openings. For example, certain people have an allergic reaction to contact with metals, particularly nickel alloys. In some cases metals are actually absorbed through the skin or ear tissue and result in infection and/or illness. Plastic materials used in earrings have also been a source of irritation and discomfort due to their relatively coarse surface textures and molding seams and/or tissue permeability.

Bacterial infections within the earlobe opening are another commonly experienced problem. Perspiration and environmental moisture can carry surface bacteria, dirt, and microscopic debris to the earlobe opening. Where hygroscopic materials are used at the portion of the earring that penetrates that opening, the moisture-borne bacteria and debris can actually be transported to the interior of the earlobe. Especially when the ear tissue has been recently pierced, this can cause painful infections which persist even after the earring is removed. In addition, infections can also result within the earlobe opening directly from metabolic reactions with respect to the earring material.

Further, irritating discomfort has sometimes been caused by the earring construction itself. Optimally, the portion of the earring penetrating the earlobe should be as small as possible so as to minimize tissue exposure and supporting compression about the opening, especially immediately after the ear is pierced. It is also important that sharp edges, which could cut or scrape the skin surface, be avoided in both the earring support structure and the attached ornament.

These concerns and ones related to skin-irritating articles such as jewelry have been long felt, and numerous unsuccessful attempts have been made to provide universally acceptable jewelry. For example, with respect to pierced earrings, it has been suggested to plate the “post” portion of the earring penetrating the earlobe with generally non-allergenic material, such as gold. However, not only does such plating commonly wear-off, gold is rarely plated in its pure form because of its relative softness. Gold-nickel alloys are commonly used, but even that nickel can be the source of an allergic reaction.

Posts formed from pure gold are often prohibitively expensive and not strong enough to properly support the ornament unless considerably thickened. Again, however, such posts are often uncomfortable because of the extra tissue compression they require within the ear opening. Further, a significant number of people suffer an allergic response to tissue contact with any metallic substance.

Even where the post construction is comfortable, earrings can cause irritation of the exterior skin surface for the same and for different reasons. Materials sensitivity can require that even the ornament base attached to the post be formed from a non-allergenic material. This can significantly increase the earring cost and make it difficult to attach an ornament to that base. Also, where skin contact with the ornament material would cause discomfort, the base must be large enough and carefully configured to prevent such contact.

Shields and sleeves of non-allergenic materials have been proposed which slide over the post and/or the base of otherwise unusable materials. While these have been satisfactory solutions for some people, others have found that base shields do not completely prevent moisture borne migration from the underlying metals to the skin or ear tissue. Also, sleeves are by necessity larger than the underlying post and can cause uncomfortable tissue compression within the earlobe opening. Further, sleeves are typically marketed such that they are cut to fit and mounted by the user and thereby require considerable care to avoid leaving sharp edges to scrape or cut the skin. On the other hand, shields and sleeves that are permanently mounted to the earring by the manufacturer are significantly more expensive.

Various plastic materials have been suggested for use in earrings. However, plastics are often difficult to mold into thin, rod-like components without a considerable loss of strength and rigidity. Thus, as compared with metal posts, plastic posts are often considerably thicker and thereby cause uncomfortable compression of the earlobe opening tissue. Also, in some molding processes a surface seam remains on the finished product which can cause skin irritation. Further, it can be more difficult to secure some metal ornaments to a plastic base and many plastic pigments are toxic with prolonged tissue contact.

Regardless of the substrate type, plastic materials themselves can also be absorbed through the skin to cause inflammation and infection. While many non-hygroscopic plastic materials are known, not all of these are non-allergenic, inexpensive and readily molded into thin and complex parts.

Finally, it has been suggested that infection and irritation can be avoided by gradually conditioning the skin. For example, when an earlobe is initially pierced, earrings having very thin posts are used and are temporarily coated with medication. After a while, larger earring posts and unmedicated earrings are substituted. However, this approach can be relatively expensive since a duplicity of earrings must be acquired, some of which are specially formed and medicated. Also, success is not ensured; allergic reactions and infection can occur when the medication ceases.

What is desired is a coating for use on a skin-contacting article and a coated skin-contacting article that will prevent skin irritation and simultaneously provide medicaments, which can thwart skin irritations or treat skin irritants already present on the skin.

### SUMMARY OF THE INVENTION

The present invention is a coating for use on a skin-contacting article and a coated skin-contacting article for preventing skin irritations and providing medication. The coating is any film-forming material which contains medicaments such as antimicrobials. The medicaments used herein combat infections, allergies, inflammation and itching. The coating is applied to articles that contact the skin, for example jewelry, including pierced jewelry. Once applied, the coating dries as a film on the applied surface.

### DETAILED DESCRIPTION OF THE INVENTION

The present invention is directed to a skin-contacting article bearing a medicated coating. The coating comprises film-forming materials such as natural or synthetic resins. Collodion is also a useful film former. Medicaments such as antibiotics and anti-inflammatories, i.e., steroids can be added to the coating material. The coated articles prevent skin irritations that often arise during wearing of or piercing with jewelry of uncoated metals. Skin irritations may arise from an allergic reaction between the skin and the metal and can lead to aggravated problems such as cysts or abscesses. The coating of the present invention prevents skin irritation by providing a barrier between the metal surface and the skin and can provide medication for already irritated skin.

Suitable resinous film-forming materials used herein include thermoplastic polymers such as, but not limited to, polycarbonates, particularly aromatic polycarbonates, polyacetals, polyarylates, polyarylene ethers, polyphenylene ethers, polyarylene sulfides, polyphenylene sulfides, polyimides, polyamideimides, polyetherimides, polyetherketones, polyaryletherketones, polyamides, polyesters, liquid crystalline polyesters, polyetheresters, polyetheramides, polyesteramides, and polyestercarbonates.

Suitable additional polymers include homo- and copolymeric aliphatic olefin and functionalized olefin polymers (which are homopolymers and copolymers comprising structural units derived from aliphatic olefins or functionalized olefins or both), and their alloys or blends. Illustrative examples include, but are not limited to, polyethylene, polypropylene, thermoplastic polyolefin (“TPO”), ethylene-propylene copolymer, poly(vinyl chloride), poly(vinyl chloride-co-vinylidene chloride), poly(vinyl fluoride), poly(vinylidene fluoride), poly(vinyl acetate), poly(vinyl alcohol), poly(vinyl butyral), poly(acrylonitrile), acrylic polymers such as those of (meth)acrylamides or of alkyl (meth)acrylates such as poly(methyl methacrylate) (“PMMA”), and polymers of alkenylaromatic compounds such as polystyrenes, including syndiotactic polystyrene.

Blends of any of the foregoing polymers may also be employed herein as film-forming material. These include blends of thermoset polymers with thermoplastic polymers such as polyphenylene ether, polyphenylene sulfide, polysulfone, polyetherimide or polyester. The thermoplastic polymer is typically combined with thermoset monomer mixture before curing.

Another suitable film-forming material of the present invention is Collodion or Flexible Collodion. Collodion is a solution of 4 g. of pyroxylin (chiefly nitrocellulose) in 100 ml of a mixture of 25 milliliters alcohol and 75 milliliters ether. Collodion is a colorless or slightly yellow, clear or slightly opalescent syrupy liquid. The Flexible Collodion comprises simple Collodion with the addition of camphor and 3% castor oil (by weight). Flexible Collodion is slightly yellow and is a syrupy liquid which contains 67% ether and about 22% absolute alcohol by volume. When the Collodion or Flexible Collodion dries it leaves a tough and colorless film. Collodion with or without an additional medicament as described below is a particularly useful coating material to prevent allergenic reaction between metal and skin.

Regardless of what film-forming materials are used, the coating may have incorporated therein dermatologically active components which can be used to readily and effectively treat a variety of adverse skin conditions arising from contacting skin-irritating substances with the skin. The film-forming material contains one or more medicaments (active ingredients) for application onto the desired substrate. In this invention, topical actives including antibiotics and steroids are incorporated into the film-forming material.

Medicaments used herein are employed to thwart infections, allergies, inflammation and itching. Suitable medicaments include antimicrobial substances that kill or slow microbial growth. One class of antimicrobials include antibiotic drugs. Antibiotics are relatively harmless to humans and are often used to treat infections. Specific antibiotics include, but are not limited to:

Antibacterial antibiotics:
Aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).
Synthetic antibacterials:
2,4-Diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, n²-formylsulfisomidine, n⁴-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibomol).
Antifungal antibiotics:
Polyenes (e.g., amphotericin b, candicidin, dermostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin).
Synthetic antifungals:
Allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

### Antineoplastic:

Antibiotics and analogs (e.g., aclacinomycins, actinomycin f¹, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites (e.g. folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur).

The polymyxin antibiotics, most notably polymyxin B and polymyxin E (also known as colistin) are cyclic polypeptide compounds produced by certain strains of Bacillus polymyxa. Polymyxins serve to damage cytoplasmic membranes of bacteria. Polymyxins are common components in topical ointments.

The antibiotic, regardless of type, may be present in the coating in a range about 0-5 w.t. %, preferably in a range about 0.5-4 w.t. %, and more preferably in a range about 1-3 w.t. %.

The coating may also include components to alleviate itching, relieve pain and/or inflammation such as a steroid/anti-inflammatory. Steroids are known to have many functions. The steroids having anti-inflammatory effect employed in the present composition are also used to reduce swelling, pain as well as inflammations. Corticosteroids are a natural or synthetic steroid that have anti-inflammatory properties. Synthetic corticosteroids mimic or augment the effects of natural corticosteroid hormones that are produced by adrenal glands. Corticol is a naturally produced example of a corticosteroid.

Steroid compounds used in the invention to provide an anti-inflammatory effect may include cortisone, hydrocortisone, fluxinanide, fluoromethalone. Other anti-inflammatory steroid agents include, but are not limited to: triamcinolone and its derivatives (particularly the diacetate, hexacetonide, and acetonide), betamethasone and its derivatives (including particularly the dipropionate, benzoate, sodium phosphate, acetate, and valerate), dexamethasone and its derivatives (particularly the dipropionate and valerate), flunisolide, prednisone and its derivatives (particularly its acetate), prednisolone and its derivatives (particularly its acetate, sodium phosphate and tebutate), methylprednisolone and its derivatives (particularly its acetate and sodium succinate), fluocinolone and its derivatives (particularly the acetonide), diflorasone and its derivatives (particularly the diacetate), halcinonide, desoximetasone (desoxymethasone), diflucortolone and its derivatives (particularly the valerate), flucloronide (fluclorolone acetonide), fluocinonide, fluocortolone, fluprednidene and its derivatives (particularly the acetate), flurandrenolide (flurandrenolone), clobetasol and its derivatives (particularly the propionate), clobetasone and its derivatives (particularly the butyrate), alclometasone, flumethasone and its derivatives (particularly the pivalate), fluocortolone and its derivatives (particularly the hexanoate), amcinonide, beclometasone and its derivatives (particularly the dipropionate), fluticasone and its derivatives (particularly the propionate), difluprednate, and desonide.

The preferred anti-inflammatory steroid used herein is hydrocortisone. Hydrocortisone is a well-known chemical that may be produced either by the human adrenal cortex, or synthetically. It is often used in the treatment of a wide array of ailments, including inflammations, allergies and arthritis. Hydrocortisone is used in many topical preparations as a treatment for temporary relief of itching associated with minor skin irritation, inflammation and rashes due to eczema, insect bites, poison ivy, poison oak, poison sumac, soaps, detergents, cosmetics, seborrheic dermatitis, psoriasis and itching in the genital and anal areas of the body. Hydrocortisone speeds up the healing process in wounds or sores that are especially prone to swelling. Hydrocortisone is also helpful in applications where the sores are not particularly prone to swelling. The steroid may be present in the coating in a range about 0-5 w.t. %, preferably in a range about 0.1-4 w.t. %, and more preferably in a range about 0.2-2 w.t. %.

The invention is particularly useful for coating jewelry, including pierced jewelry. To coat the jewelry, the film-forming composition likely needs to be contained within a carrier solvent for application. In general, organic solvents are useful for providing the synthetic resin in liquid form and allowing ready application to the jewelry. Specific examples of organic solvents are alcohols such as methyl alcohol, ethyl alcohol, n- or iso-propyl alcohol, n- or iso-butyl alcohol and diacetone alcohol; ketones such as acetone, methyl ethyl ketone, methyl propyl ketone, methyl butyl ketone, methyl amyl ketone, methyl hexyl ketone, diethyl ketone, di-isobutyl ketone, cyclohexanone, methyl cyclohexanone and acetyl acetone; hydrocarbons such as benzene, toluene, xylene, cyclohexane and methoxy benzene; acetic acid esters such as ethyl acetate, n- or iso-propyl acetate, n- or iso-butyl acetate, ethylbutyl acetate and hexyl acetate; halides such as methylene dichloride, ethylene dichloride and monochloro-benzene; ethers such as isopropyl ether, n-butyl ether, dioxane, dimethyl dioxane and tetrahydrofuran; polyhydric alcohols and derivatives thereof such as ethylene glycol, methyl cellosolve, methyl cellosolve acetate, ethyl cellosolve, diethyl cellosolve, cellosolve acetate, butyl cellosolve, butyl cellosolve acetate, methoxy-methoxy ethanol, diethylene glycol monomethyl ether, diethylene glycol dimethyl ether, diethylene glycol methyl-ethyl ether, diethylene glycol diethyl ether, propylene glycol, propylene glycol monomethyl ether, propylene glycol monomethyl ether acetate, propylene glycol monoethyl ether, propylene glycol monoethyl ether acetate, propylene glycol monobutyl ether and 3-methyl-3-methoxy butanol; and special solvents such as dimethylsulfoxide and N,N-dimethyl-formamide, which may suitably be used alone or in any combination.

If the film-forming composition is water-soluble or -dispersable, water can be used as the carrier for the film-forming composition. Surfactants may be needed to readily disperse the film-forming composition in water. Combinations of water with organic solvents may also be used. Instead of a solvent as a carrier for the film-former, some film-formers may be useful in molten form without use of a solvent.

Once dispersed in the liquid carrier, the film-forming composition can be used via an easy-to-use dispensing package/device to facilitate dispensing of the coating on the intended article. For example, the composition may be dispensed using common applicators such as a brush, roll or eye dropping apparatus, spray bottles, sheets retaining the coating in liquid form, etc. Thus the coating can be applied to the jewelry substrate or other skin-contacting article, preferably after cleaning, by dipping, swabbing, wiping, spraying, or a combination of theses and other methods.

Pierced earrings are one type of jewelry substrate that can cause skin irritations upon wearing. Often the post of the earring that pierces the skin has caused skin allergies, inflammation and infections. Likewise, other skin-contacting portion of the earring such as the earring backing and the inner-rear surface of the front of the earring have caused allergic reactions, inflammation and infections to the portions of the skin it contacts. The present invention provides relief for such problems by application of the coating on these specific substrate surfaces.

With regard to application via spray bottles, aerosol container or a non-aerosol spray device may be used. Said spray means is any of the manually activated, preferably “trigger-type,” means for producing a spray of liquid droplets as is known in the art. Typical spray means are disclosed in U.S. Pat. No. 4,082,223, Nozawa, issued Apr. 4, 1978; U.S. Pat. No. 4,161,288, McKinney, issued Jul. 17, 1979; U.S. Pat. No. 4,558,821, Tada et al., issued Dec. 17, 1985; U.S. Pat. No. 4,434,917, Saito et al., issued Mar. 6, 1984; and U.S. Pat. No. 4,819,835, Tasaki, issued Apr. 11, 1989, all of said patents being incorporated herein by reference. Examples of spray bottles are those in U.S. Design Pat. No. 244,991, Weekman et al., issued Jul. 12, 1977; and U.S. Design Pat. No. 275,078, Wassergord et al., issued Aug. 14, 1984, said patents being incorporated herein by reference.

The spray device can also be one that can be adjusted to either give a liquid spray or a foam. The spray means herein are typically those that act upon a discrete amount of the coating itself, typically by means of a piston that displaces the coating and expels the coating through a nozzle to create a spray of thin liquid. After application to the surface of the article it is then dried.

Consumers may apply the coating to the desired skin-contacting article, e.g. pierced earring, via brushing or swabbing. For example, the composition may be semi- viscous, gel-like or liquid disposed in a closed container. The container may have a screw-on cap or otherwise tightly closing cap bearing an applicator on its inner side. The applicator has a long shaft bearing bristles or a swab at its distal end. The applicator is long enough to reach more than half-way inside the container without touching the bottom of the container. Thus, the applicator is already immersed in the coating.

In use, the consumer would unscrew the cap from the container wipe-off any excess amounts of coating already on the applicator and apply the coating via brushing or swabbing on the pierced earring. Any number of layers may be applied to the article. Drying time should be allotted either between or after the application of layer(s) to form the coating into a dry film.

In another method of use, the consumer may be provided the coating as a liquid in a tub. The consumer would dip the pierced earring or other jewelry into the coating, remove any excess coating from the substrate and allow time to dry to form the dry film. In yet another method, a single-use wipe retaining the coating could be used to apply the coating on the jewelry. The wipe could be a 1″×1″ cloth saturated with the coating. The wipe is disposed in an individual, sterile wrapper that is ripped open to expose the swab. The wipe is then wiped, rubbed, dabbed or otherwise spread on the jewelry, and finally allowed to dry to form the dry film.

After application of the coating, by the methods disclosed above, the article is dried to form the irritant-free article. In general, the drying of the coating is conducted using air at any temperature. In some cases, good results would be obtained by removing any humidity in the air for drying.

The coating may initially have any viscosity when applied directly onto the substrate, but after the coating dries, it forms a protective film close to the article's surface to maintain contact of the active ingredients on the skin and prevent removal of the active ingredients from the article.

What is claimed is:

1. Coated jewelry comprising a jewelry substrate, a layer of a dried film coated onto said substrate, said film containing a film-former, and a medicament.

2. The coated jewelry of claim 1, wherein the film-former is collodion.

3. The coated jewelry of claim 1, wherein the film-former is a natural resin.

4. The coated jewelry of claim 1, wherein the film-former is a synthetic resin.

5. The coated jewelry of claim 1, wherein the medicament is an antibiotic.

6. The coated jewelry of claim 5, wherein the medicament is bacitracin or polymyxin.

7. The coated jewelry of claim 1, wherein the medicament is a steroid.

8. The coated jewelry of claim 7, wherein the steroid is hydrocortisone.

9. The coated jewelry of claim 1, wherein the medicament is a combination of a steroid and an antibiotic.

10. The coated jewelry of claim 9, wherein the steroid is hydrocortisone.

11. The coated jewelry of claim 1, wherein the jewelry is for piercing the body.

12. The coated jewelry of claim 11, wherein the piercing is earrings.

13. Coated jewelry comprising a jewelry substrate, a layer of a dried collodion film coated onto said substrate.

14. The coated jewelry of claim 13 wherein said jewelry substrate is pierced earrings.

15. A method for producing irritant-free jewelry comprising the steps of applying a coating comprising a film-former and a medicament onto jewelry and drying the coating to produce a dried film coated on said jewelry.

16. The method of claim 15, wherein the film-former is collodion.

17. The method of claim 15, wherein the film-former is a resin.

18. The method of claim 15, wherein the medicament is an antibiotic.

19. The method of claim 15, wherein the medicament is hydrocortisone.

20. The method of claim 15, wherein the coating is applied by rolling, dipping, dripping, spraying, swabbing and wiping said coating, as a liquid onto said jewelry.
